(19) United States
(12) Patent Application Publication
(10) Pub. No.: US 2020/0043596 A1
(43) Pub. Date: Feb. 6, 2020

(54) STREAMLINED NUTRITION TRACKING SYSTEM

(71) Applicant: Ryan Koretoff, Santa Monica, CA (US)

(72) Inventor: Ryan Koretoff, Santa Monica, CA (US)

(21) Appl. No.: 16/530,575

(22) Filed: Aug. 2, 2019

Related U.S. Application Data
(60) Provisional application No. 62/714,924, filed on Aug. 6, 2018.

(51) Int. Cl. G16H 20/60; G16H 50/30

(52) U.S. Cl. CPC G16H 20/60; G16H 50/30

(57) ABSTRACT

A streamlined nutrition tracking application can provide user devices with access to a user interface that enables a user to input simplified information corresponding to a food or beverage the user consumes. This simplified input can be made possible in part by a backend system's streamlined nutrition tracking component, which can simplify nutritional information obtained from an USDA (Food and Drug Administration) database. A machine learning component can improve the accuracy of users' simplified inputs over time, based on the aggregated inputs of many users. The application can access the machine learning component to request an adjustment of the user food consumption input based on aggregated machine learning of user food consumption inputs of a plurality of users. The application can output nutritional performance data to the user based on the machine learning, the user's basic metabolic rate, and/or lifestyle factors, as a graph or chart that indicates nutritional consumption of the user over time.

### CROSS-REFERENCE TO RELATED APPLICATIONS

This application claims the benefit of priority to U.S. Patent Application No. 62/714,924, filed Aug. 6, 2018, STREAMLINED NUTRITION TRACKING SYSTEM, which is hereby incorporated by reference herein in its entirety.

### FIELD OF THE DISCLOSURE

The present disclosure relates to nutrition tracking applications.

### BACKGROUND

Nutrition tracking applications exist for tracking calories consumed, types of foods eaten, and so forth. However, many nutrition tracking applications have defects that inhibit their usefulness. For instance, nutrition tracking applications are often overburdened with features that overwhelm users. As an example, a user eating a meal may wish to enter the details of that meal into the nutrition tracking application and mirror that of his/her consumption. The application, however, may present a bewildering array of options, too detailed for a user to easily and quickly select from. As a result, users may give up nutrition tracking and consequently struggle to change their eating habits.

### SUMMARY

A streamlined nutrition tracking system can include: a memory device programmed with computer-executable instructions corresponding to a streamlined nutrition tracking application; a hardware processor able to execute the computer-executable instructions so as to execute the streamlined nutrition tracking application on a user device, the application able to: receive an input of a user's demographic parameters; calculate a basic metabolic rate based on the input of the user's demographic parameters; calculate an adjusted metabolic rate based on the input of the user's lifestyle parameters and norms; output a nutritional user interface that comprises streamlined nutritional options derived from a database and one or more fields that enable the user to provide user food and/or beverage consumption input based on the streamlined nutritional options; receive, from the nutritional user interface, user food and/or beverage consumption input based on the streamlined nutritional options; request an adjustment of the user food and/or beverage consumption input based on aggregated machine learning of user food and/or beverage consumption inputs of a plurality of users and based on aggregated user specific performance against user set benchmarks; and output nutritional performance data to the user based on the adjusted metabolic rate and the adjustment of the user food and/or beverage consumption input, the nutritional performance data including a graph or chart that indicates nutritional consumption of the user over time.

The user's demographic parameters can include at least one of: gender, weight, height or age.

The user's lifestyle parameters and norms can include an estimated average level of exertion or frequency of physical activity.

The user's lifestyle parameters and norms can include data associated with at least one monitoring device such as a smart weight scale, physical activity monitor, glucose monitor, or other biometric readers.

The application can be configured to receive a user's current fitness goal(s). The fitness goal(s) can include body building, maintenance, or fat loss, which can be updated or changed at the user's discretion.

The application can be able to determine one or more nutrition benchmarks based on the user's current fitness goal. The user set benchmarks can include one or more updated nutrition benchmarks based on user-generated modifications to the one or more nutrition benchmarks. The user-generated modifications can be limited by the basic metabolic rate or adjusted basic metabolic rate.

The graph or chart that indicates nutritional consumption can include a moving average nutrition against the user set benchmarks.

The application can be able to analyze the beverage or food consumption input to determine a projection of the user's body composition.

The adjustment to the food or beverage consumption input can include a change in the type of food or beverage or a change in a portion size of the food or beverage.

The application can be able to: receive a user indication of an unknown food or beverage; request information associated with the unknown food or beverage; and determine an identity of the unknown food or beverage based on the requested information.

The application can be able to determine a target caloric burn per user workout based on the adjusted basic metabolic rate.

A method for streamlined nutrition tracking can include: receiving an input of a user's demographic parameters; calculating a basic metabolic rate based on the input of the user's demographic parameters; calculating an adjusted metabolic rate based on the input of the user's lifestyle parameters and norms; outputting a nutritional user interface that comprises streamlined nutritional options derived from a database and one or more fields that enable the user to provide user food and/or beverage consumption input based on the streamlined nutritional options; receiving, from the nutritional user interface, user food and/or beverage consumption input based on the streamlined nutritional options; requesting an adjustment of the user food and/or beverage consumption input based on aggregated machine learning of user food and/or beverage consumption inputs of a plurality of users and based on aggregated user specific performance against user set benchmarks; and outputting nutritional performance data to the user based on the adjusted metabolic rate and the adjustment of the user food and/or beverage consumption input, the nutritional performance data comprising a graph or chart that indicates nutritional consumption of the user over time.

The method can include receiving a user's current fitness goal, which can be updated or changed at the user's discretion.

The method can include determining one or more nutrition benchmarks based on the user's current fitness goal.

The method can include analyzing the beverage or food consumption input to determine a projection of the user's body composition.

The method can include receiving a user indication of an unknown food or beverage; requesting information associated with the unknown food or beverage; and determining an identity of the unknown food or beverage based on the requested information.

The can include determining a target caloric burn per user workout based on the adjusted basic metabolic rate.

For purposes of summarizing the disclosure, certain aspects, advantages, and novel features have been described herein. Of course, it is to be understood that not necessarily all such aspects, advantages, or features will be embodied in any particular embodiment.

### BRIEF DESCRIPTION OF THE DRAWINGS

FIG. 1 illustrates an example computing environment for tracking nutrition in a streamlined manner.

FIG. 2 illustrates an example basic metabolic rate (BMR) calculation.

FIG. 3 illustrates example fitness goals.

FIG. 4 illustrates example benchmark nutrition information that may be part of a user dashboard.

FIG. 5 illustrates an example macronutrient management analysis.

FIG. 6 illustrates an example macronutrient management analysis over a period of time.

FIG. 7 illustrates example nutritional information that may be included in an example tracking application.

FIG. 8 illustrates an example aspect of a user interface for an example tracking application for tracking food intake.

FIG. 9 illustrates an aspect of a user interface for an example tracking application for tracking food portion sizes.

FIG. 10 illustrates another aspect of a user interface for an example tracking application for tracking food portion sizes.

FIG. 11 illustrates an example of altering a benchmark variance calculation.

FIG. 12 illustrates another example of altering a benchmark variance calculation.

### DETAILED DESCRIPTION

### Overview

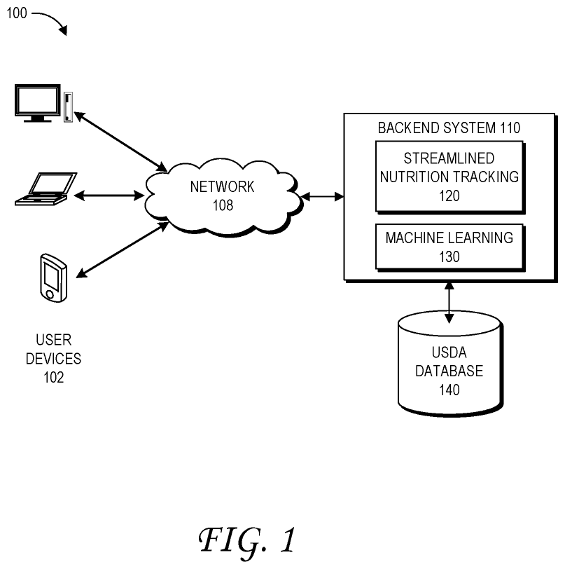

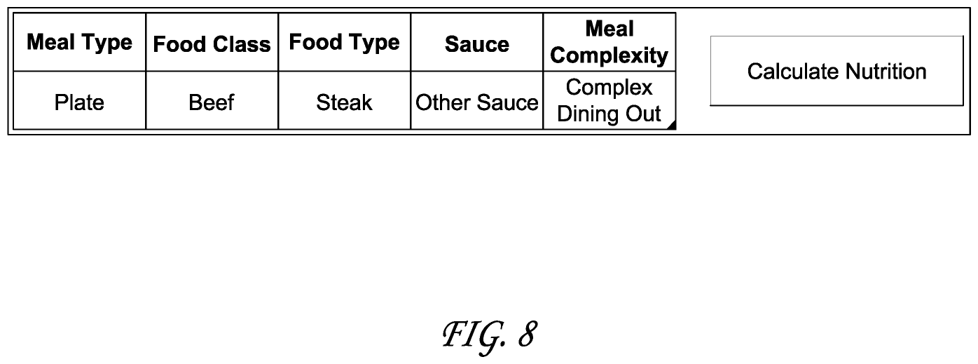

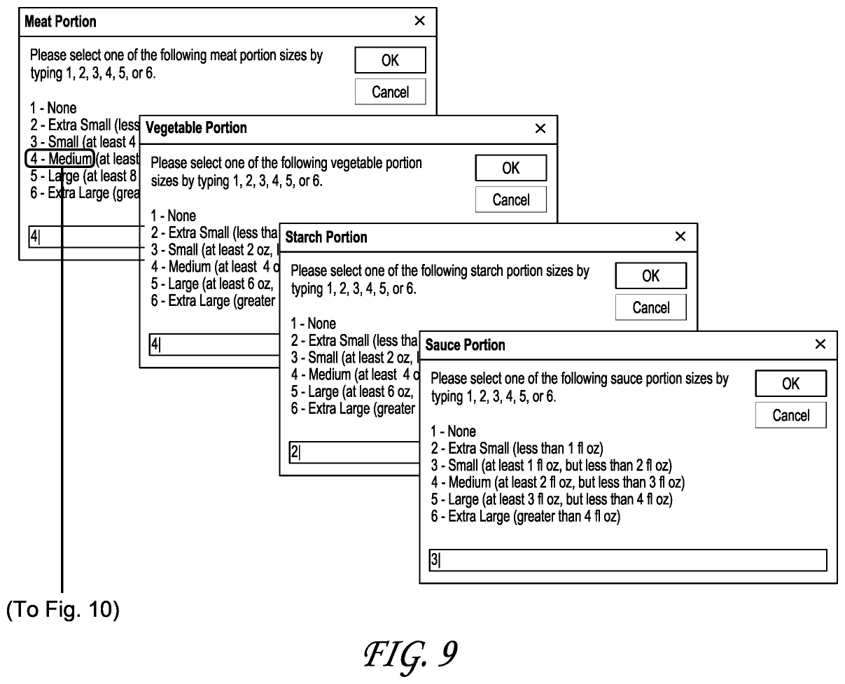

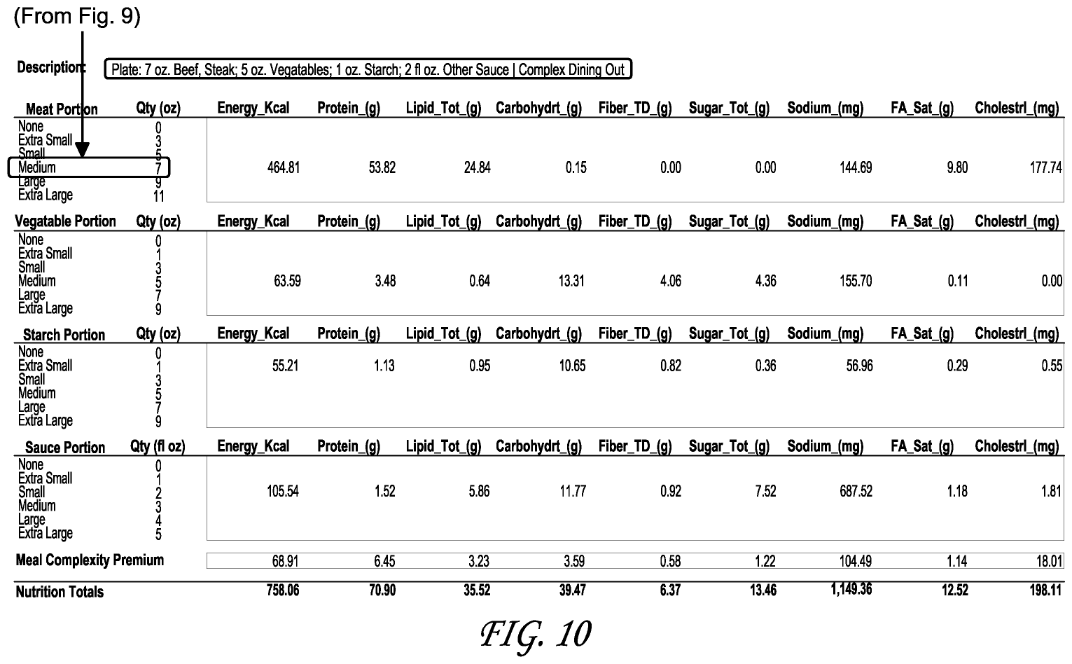

This disclosure describes example systems and methods for nutrition tracking that are streamlined, facilitating wide user adoption. The systems and methods described herein can be used, for example, in facilitating a user's fitness goals, educating a user on fitness habits, or treating a user for fitness-related lifestyle diseases. These systems and methods can be implemented in a variety of ways and can be manual, automated, or semi-automated. For instance, referring to FIG. 1, an example computing environment 100 is shown for tracking nutrition in a streamlined manner. User devices 102 can access a mobile application or web application (or simply “application”; not shown), which can communicate with a backend system 110 over a network 108 (such as the Internet). The application can output a user interface that enables a user to input simplified information corresponding to food or beverage the user consumes (see, for example, FIGS. 8 and 9). This simplified input can be made possible in part by the backend system's streamlined nutrition tracking 120 component, which can simplify nutritional information obtained from a centralized database, such as the USDA (United States Department of Agriculture) database 140. Example USDA food composition databases can be accessed at https://ndb.nal.usda.gov/ndb/.

Further, a machine learning component 130 can improve the accuracy of users' simplified inputs over time, based on the aggregated inputs of many users. The application can access the machine learning component 130 to request an adjustment of the user food consumption input based on aggregated machine learning of user food consumption inputs of a plurality of users. The application can output nutritional performance data to the user in the form of a graph or chart that indicates nutritional consumption of the user over time. The machine learning component 130 can also improve the user interfaces described herein. For example, user interfaces on mobile displays have a limited real estate. Too many options can clutter the user interface. Accordingly, the improvement in user interface can include how to display relevant information in the limited real estate of a computing device, such as a mobile device.

The user devices 102 can be mobile phones, smartphones, tablets, phablets, laptops, desktops, or the like. More generally, the user devices 120 can be any device with a hardware processor, memory, and a display (or connectable to a display). The backend system 110 can be implemented as one or more physical or virtual servers, which may be geographically dispersed or co-located. The backend system 110 may be implemented in a cloud computing platform. The database 140 can be a local copy of the USDA's central database and may be stored in physical computer storage or it can be accessed in a cloud computing environment.

Specific examples of these systems and methods are described in detail below.

### Example Client Intake and BMR Calculation

The Client Intake can include an email and password for login purposes and basic demographic information used to calculate the individual's basic metabolic rate (BMR). Examples of such basic demographic information include, but are not limited to, gender, weight, height, and age. The user's basic BMR can be calculated based on the Harris-Benedict equation.

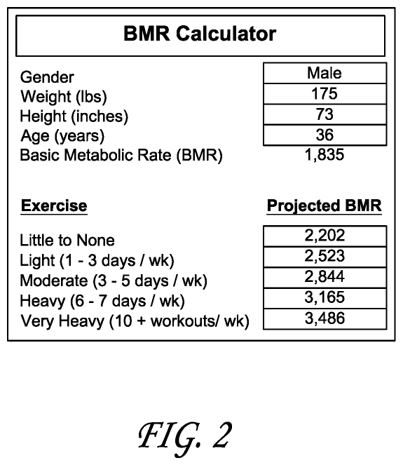

Upon intake, the client can be prompted to rate his or her weekly estimated average level of exertion or frequency of physical activity. The goal of this calculation can be to guide the user's daily caloric need to maintain his or her current weight by applying a premium to the basic BMR. (See FIG. 2.)

The exercise parameters displayed herein can improve with accuracy. First, they can tighten up with a short client intake survey that asks about the user's general lifestyle. For example, one question of the survey may ask about the user about his or her type of job (for example, athlete v. clerical), while another question may ask about the frequency, duration, and intensity of the user's workout routine (for example, 2-3 times per week of strenuous exercise for an average of 45 minutes per workout). Second, machine learning on user perceptions of physical activity can improve the accuracy of the BMR premium to arrive at a total daily caloric need. For example, one user's perception of strenuous exercise may be significantly different than that of another, even though they share the exact same demographics. Finally, when coupled with lifestyle monitoring devices such as smart weight scales, physical activity monitors (for example, pedometer, heart rate monitor, etc.), and biometric readers (e.g. glucose monitor), machine learning can further personalize this BMR premium to provide additional guidance for the user to meet his or her current fitness goals.

Having an intuitive and personalized intake survey can lead to more accurately estimating the needed caloric burn required to reach the user's fitness goal(s). A user's BMR premium can be accurately estimated by analyzing a collection of factors, including, but not limited to, one's general lifestyle or leisure activities, occupation, and physical exercise. Comparing one's adjusted BMR (user's BMR plus its respective BMR premium) with his or her average consumption can yield a recommended target caloric burn per workout. Equipping users with such information can further guide them in committing to lifestyle adjustments that can lead to obtaining their fitness goals.

Coupling machine learning on user perceptions of physical activity and lifestyle monitoring devices can create an actual data spectrum whereby a more accurate BMR premium can be calculated or automatically adjusted. The equation can be a function of a user's reported physical activity and data collected from such devices to support actual user physical activity. The results can normalize as more users sync such devices with the application, consistent with the law of large numbers. For example, if 1,000 people that share some or all of the same demographic data reported that they exercise 3 times per week, and some or all of these individuals have a heartrate monitor synced with the application, the system can calculate or more accurately estimate an average caloric burn per workout. With that information, the system can systematically apply those 3 days of extra caloric burn to the base BMR to arrive at an accurate and personalized BMR premium.

Although syncing with lifestyle monitoring devices can significantly enhance the maintenance of these profile variables, the fields can remain manually modifiable in the user profile as they can temporarily change from time to time (for example, training for a marathon).

### Example Fitness Goals

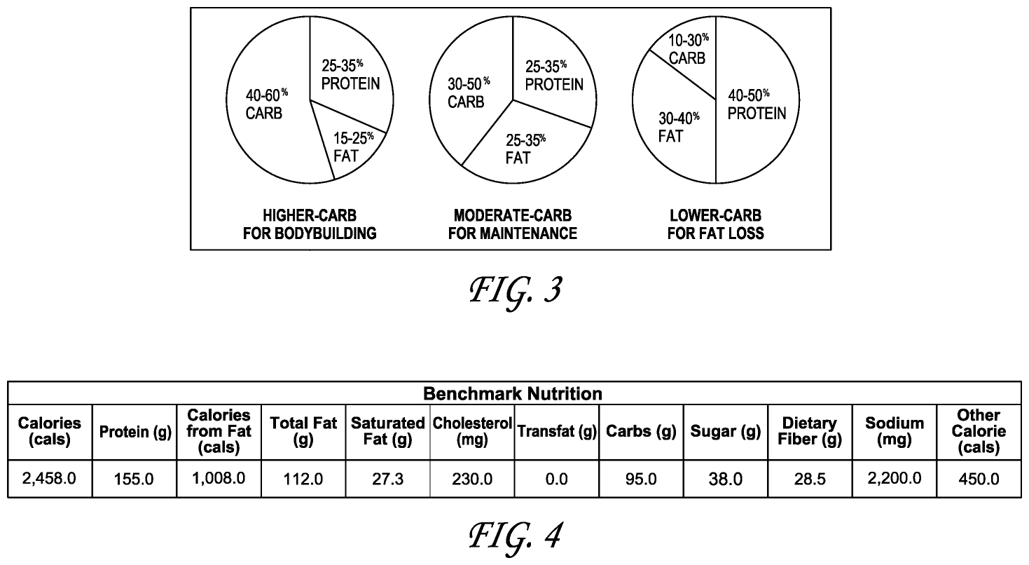

As part of the user profile setup and maintenance, the user can be asked to provide information leading to his or her fitness goals. Although fitness goals can be personalized further and can pertain to a combination of objectives, there are three basic fitness goals in this example: body building, maintenance, and fat loss. (See FIG. 3.) Others are possible. For example, fitness goals can be created or updated with extended application use and profile refinement. While the BMR calculation can attempt to target the user's daily caloric need for weight maintenance, the fitness goals can attempt to guide the user on the types of calories or nutrition needed to more effectively obtain his or her current fitness goals. These selections can be stored and remain modifiable in the user profile as they can change over time.

After combining the user's BMR and current fitness goals, the user can be prompted to customize his or her benchmark daily nutrition. Although benchmark ranges (for example, 150-160 grams of protein) can be determined by the individual's fitness goals and BMR, the user can have overwriting abilities in his or her profile to further customize the default settings within set parameters controlled by his or her selected fitness goal. Furthermore, error handling can prevent significant user modifications to the benchmark ranges without also modifying the key drivers (i.e. BMR and fitness goals).

### Example Benchmark Nutrition

The user's benchmark nutrition can be a collection of variables that can be trended against over time and can be presented throughout the main dashboard. (See FIG. 4.) Although there can be some key variables recommended for display, the user can have the ability to fully customize his or her dashboard. Some or all the variables can be tracked, so if the user wishes to add or subtract any variable(s) he or she can have full flexibility to do so at any given time.

In some embodiments, the advantage of this flexible UI can relate to a truly customize user experience. For example, some users may wish to body build; however, they may also suffer from diabetes which can present its unique challenges with respect to heightened carbohydrate and sugar consumption, as may be recommended by some body building diets. Another example can relate to some users who wish to get leaner by losing fat; however, some fat loss diets like the keto diet encourage greater fat consumption, which may present user-specific dietary sensitivities related to high cholesterol because they are genetically predisposed to cardiovascular disease. Regardless of the individual's dietary constraints, this application can accommodate every user's needs.

The USDA nutritional database can serve as the application's data resource and the nutrition variables can be limited to the variables presented in the database. Upon profile creation, the user can be provided default benchmarks that he or she can further modify or simply accept based upon the user intake questionnaire. The defaults and recommendations (some or all) can be based upon evidence-based research and globally recognized publications (for example, “The American Heart Association recommends that your daily sodium intake should be less than . . . ”). However, these default values can simply remain as recommendations that are modifiable within the user's profile settings.

### Example Macronutrient Management

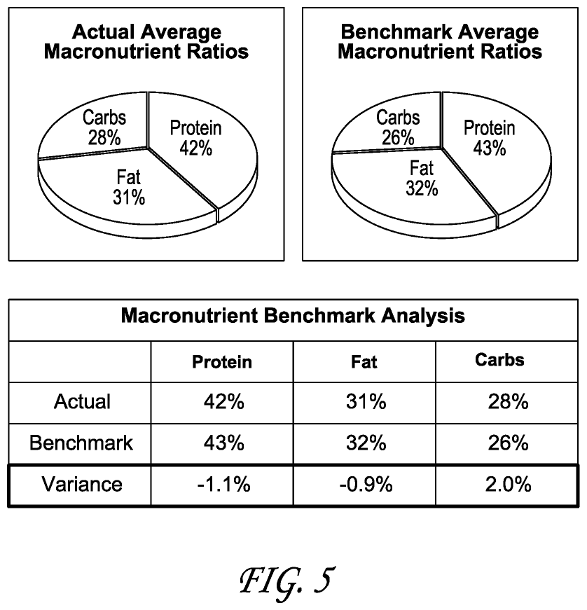

Macronutrient management can be a core of the application and its analytics. (See FIG. 5.)

Most key metrics can highlight the user's moving average nutrition against his or her benchmark nutrition. Applying psychology to this moving average can allow the users to successfully make lifestyle modifications over time. In contrast, many existing diets and nutritional tracking applications are flooded with restrictions that can present adherence challenges.

Furthermore, many people change between dieting and their normal eating habits in search for body type gains. However, if individuals were 1) presented with the ability to better understand their daily eating habits and 2) set reasonable goals with the intention to make minor lifestyle adjustments, they may find greater success with their current fitness goals. Managing a moving average can present a benefit to the user whereby he or she may not become discouraged from a single meal that falls outside of his or her diet parameters. In fact, the user may become increasingly motivated and internally competitive to make his or her moving average outperform the benchmark. In ideal cases, the users can leverage their successes and further adjust their profile benchmarks to advance their current fitness goals.

Benchmark nutritional adjustments can have a limitation parameter based upon the individual's BMR, lifestyle, and set moving averages. For example, users may not be able to increase or decrease any of their benchmarks above or below a 10% variance from the previously set benchmark. By limiting these user overrides, the user can be expected to experience positive reinforcement for exceeding his or her benchmark performance. Upon reaching these milestones, the user can receive a congratulatory notification from the application and can be allowed to further adjust his or her benchmark settings to create new goals.

If the application can be coupled with lifestyle monitoring devices, the default benchmark parameters can be set to a similarly set periodic reading from the last device installation. This setting can allow the various devices to sync and communicate with the application. However, if the user exceeds his or her benchmark performance in less than this period window, the application can reward the user with a congratulatory notification and the increased freedom to further modify his or her profile benchmarks within the system variance parameters.

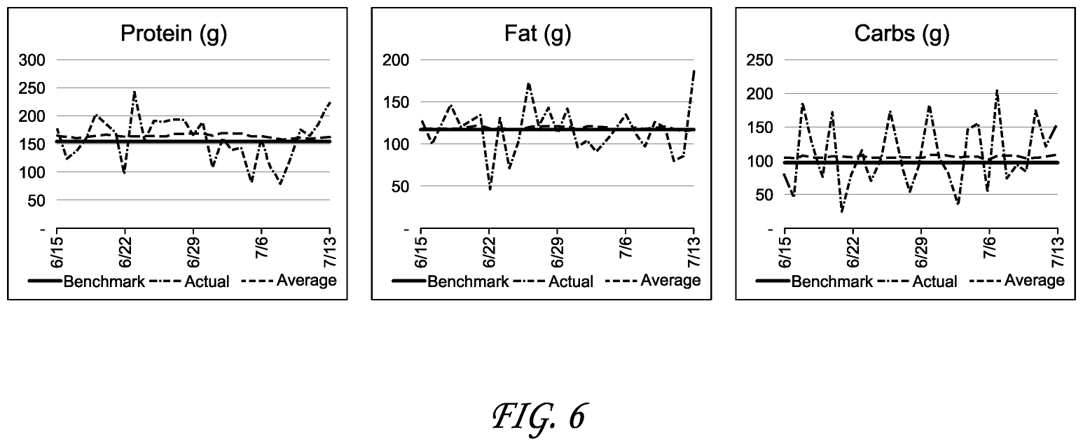

By applying the premise that anyone's daily caloric intake can be centered upon the three macronutrients (proteins, fats, and carbohydrates), this resource can remain focused on its purpose of intelligent weight management. The application may or may not attempt to directly encourage better eating habits. There can be enough nutritional visualization to create self-awareness. Conversely, it can reward the user for reaching his or her benchmark nutrition goals with congratulatory notifications and the ability to further modify his or her profile benchmarks within set system parameters. (See FIG. 6.)

### Example Database Management

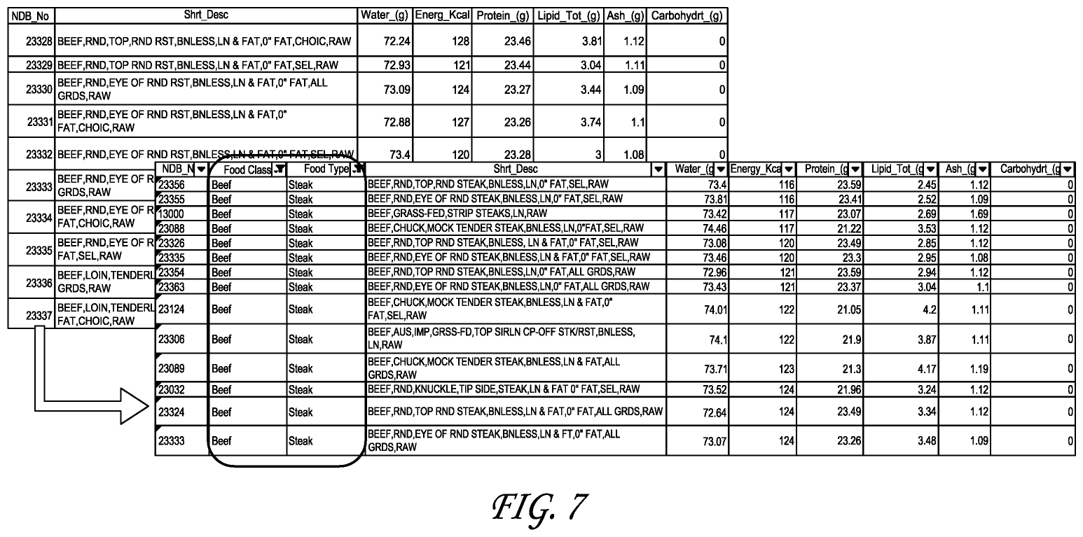

The application can calculate its nutritional information based upon the USDA nutritional database and its data can be integrated via an API. (See FIG. 7; see https://ndb.nal.usda.gov/ndb/.) When referring to “the database” herein, this specification usually is referring to a copy of the USDA database modified to include additional data in the form of metatags that can facilitate simplified or streamlined nutritional tracking.

The database can contain records with 3 primary components: 1) a label, 2) nutritional data, and 3) a baseline weight measurement (i.e. gram weight). Each record can have various metatags strategically applied to the labels that can serve as independent variables used for constructing meal nutritional calculations.

The metatags can need periodic management when there are record additions to the database. Error handling for these record additions can be minimal as the metatags can drive the calculations. In other words, new records without metatags can simply not be factored into the meal nutritional calculations until they are appropriately tagged. For example, each month the database administrator can be notified of any new records that need tagging.

In certain embodiments, an advantage of having a centralized database versus a decentralized one that may include nutritional information sourced from other databases can relate to reliability. A single centralized database can have consistent units of measurement sourced from a finite number of contributors. The methodology of database information contribution can be consistent. A decentralized database may include information from parties that exercise different protocol, thus leading to potentially significant differences in nutritional data of the exact same item. Furthermore, the consistency found within a centralized database can best accommodate personalized meal construction during the data entry process.

### Example Data Entry

This application can empower the user with the market's fastest and most convenient data entry experience. It can accomplish this by performing or leveraging one or more of the following: 1) a centralized database, 2) strategically set metatags, 3) intelligent user training, 4) machine learning on user meal size perceptions, and 5) a carefully designed front-end UI (user interface—see FIGS. 8 and 9).

Intuitive and intelligent user training can relate to tips in the form of popup windows that can occur (with “do not show again” options) when performing meal composition data entry. These can assist the data entry process with reasonably high accuracy while simultaneously training the user on his or her typical meal portion sizes. For example, a 6′1″ 180 lb. male's first may equate to an 8 oz filet and 5 oz of sauteed vegetables, while a 5′5″ 110 lb. female's first may equate to a 5 oz filet and 3.5 oz of sauteed vegetables.

The data entry process can be consistent with the application's underlying premise that lasting change occurs over time and it involves user dedication. Furthermore, it can be assumed that the user can sacrifice a reasonable margin of individual meal nutritional accuracy to gain the convenience of its data entry experience. The data entry process can differentiate this application from its market competitors. In some instances, the data entry input do not exceed more than 5 inputs as shown in FIG. 8.

Upon initial data entry, the user can be asked about the type of item he or she is consuming. The item consumed can be any plate, bowl, sandwich, wrap, snack, beverage, or any other collection of nutritional elements one could consume. The UI (see, for example, FIGS. 9 and 10) can then proceed with a series of supplemental questions, gathering additional information as appropriate to reasonably identify the item (or items) consumed. The questions can be strategically organized and synced with specific metatags to filter the database for nutritional calculations.

One example feature of the UI can involve a Meal Complexity Premium applied to the baseline nutritional calculations. The key assumption behind this premium can be that a direct relationship exists between a meal's complexity and its quantity of ingredients. Simply put, more ingredients tend to lead to more nutritional data. Furthermore, it can be also assumed that, on average, home-cooked meals tend to be less complex than those consumed when dining out. The primary advantage of this premium can relate to expediting the data entry experience by reducing the quantity of immaterial items one would need to log to arrive at an item's nutritional value.

Some or all manual data entry fields can be in the form of picklists/dropdown menus, free text, radio buttons, numbers (decimal or integer), or lookup values. Data entry can also take the form of OCR (for example, taking a picture of a receipt), voice recognition (for example, speaking to the application and telling it the items and respective quantities consumed), or integrations with other devices/applications (native or 3rd party), such as a camera.

No matter the form of assisted data entry, it can be safe to assume that the application's overall accuracy and efficiency can be enhanced to provide a more personalized user experience. For example, a smart bathroom scale and a heartrate monitor that are both synced with the application can provide real-time data on the user's Projected BMR (BMR+lifestyle premium). This can enhance the user experience by 1) empowering the user so he/she does not need to continuously manage his/her profile settings like weight or overall degree of physical activity, but also 2) more accurately measure the daily variances from his/her set nutritional benchmarks. Another example of assisted data entry may take the form of a device or application that automatically measures an item's nutritional composition. When synced with this resource, the user can effectively bypass the UI and simply confirm the data prior to its input.

As previously mentioned, machine learning can aid the data entry process by specifically assisting users with recording portion sizes. By applying the law of large numbers to users' perceptions of their portions, well-defined ingredient quantities can occur for every recording. For example, when a group of 1,000 30-yr old, 5′7″ 120 lb. women are asked to rate the portion size of a 6 oz filet mignon and 85% reply that it's “medium” sized, while the other 15% have mixed responses, it's reasonable to assume that when this demographic records a medium sized steak, they are most likely thinking of this 6 oz filet mignon.

Although the user will retain the ability to manually enter his or her nutritional information for each record, both assisted data entry and machine learning can occur with this application. They can both provide their distinct advantages that can cater to an optimized user experience and more accurate information recording. When coupled with a centralized database and an effective front-end UI that leverages strategic metatags, the user can be empowered with rapid and reliable data entry for nutritional item recording.

### Example Benchmark Variance Analysis

Example visuals contained within the application can display a form of benchmark variance analysis. These analytics can paint a picture that illustrates the user's performance towards his or her goals and may include graphs, charts, or the like.

At the center of the benchmark analyses can be the three macronutrients. Although the application can have the ability to showcase any nutritional element found within the USDA database, these three nutrient values, along with total calories (not pictured), can have the greatest overall impact on body image and can be the default measurements displayed.

The ‘Benchmark Variance’ and ‘Moving Average (Days)’ variables can control the benchmark variance analyses calculations and can be found within the user's settings. The ‘Benchmark Variance’ can be a setting that establishes boundaries on the upper and lower limits from the user's benchmark value. This setting can tighten or loosen the user's “normal” range from his or her benchmark settings. It can be thought of as a deviation from the mean, with the mean being the user's benchmarks. The ‘Moving Average (Days)’ can be a setting that controls the retroactive quantity of days that the user wants to trend against. The longer the period (or greater number of days), the smoother or more normalized the average can be. Both parameters can aid in customizing the user's experience and lend guidance towards reaching or exceeding benchmark settings.

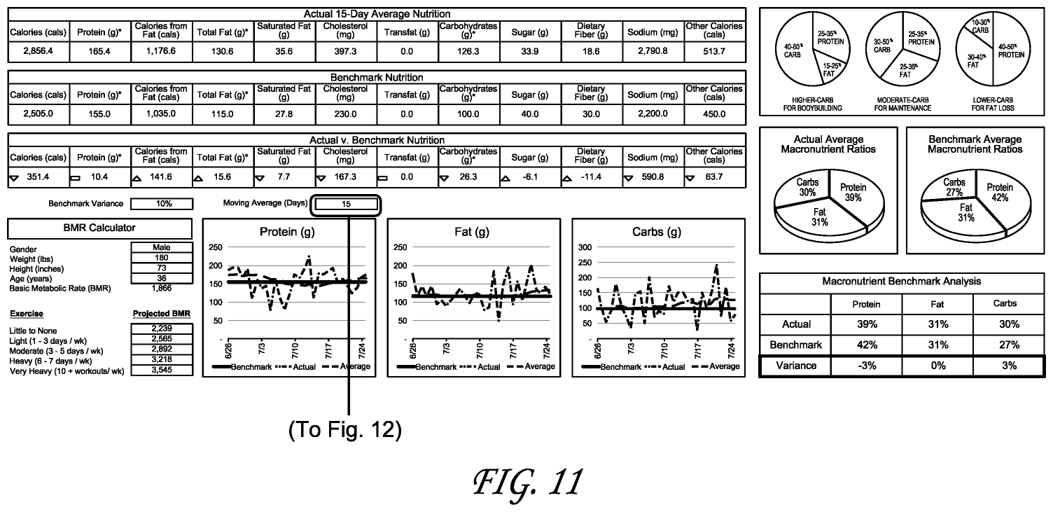

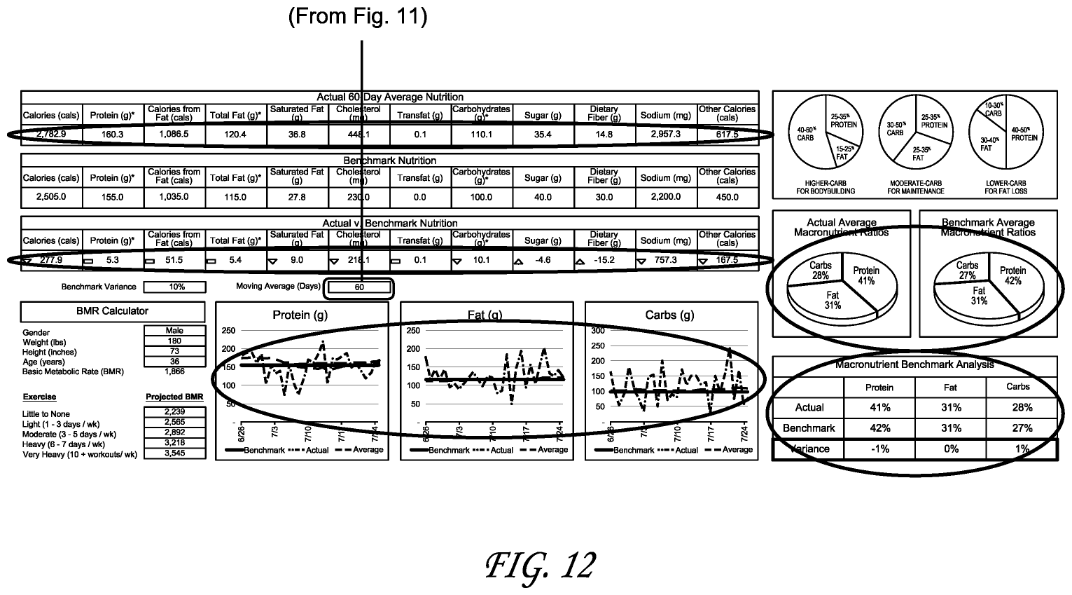

FIGS. 11 and 12 display examples of how the ‘Moving Average (Days)’ can alter the benchmark variance calculations. Similar to other rewards previously mentioned, having the ability to loosen this parameter can be a luxury for extended application use as well as a mechanism for positive reinforcement. For instance, the moving average for a new user can be much more volatile that of an older user. Examining a greater time period can bring peace of mind to an individual attempting to make dietary and lifestyle adjustments. The capability to overlook daily readings and focus on the larger picture can be critical to user success.

### Example Predictive Analytics

The application can be equipped to perform short and long-term predictive analytics that can further guide user decisions and maximize success rates. Leveraging the application's core programming, the user can have the ability to quickly and easily see the impact of a single meal or menu item on his or her daily nutritional calculations prior to its logged consumption. For example, a user may have a fitness goal of fat loss and thus might want to minimize his carbohydrate consumption. However, he may be planning to dine out and may be having difficulty choosing between sushi and steak for dinner. After performing a quick exam of his typical sushi and steak dinners, he chooses steak because he may have greater control over his carbohydrate consumption. This simple “what if” scenario can empower the user with near instantaneous information to guide meal choices.

A greater form of predictive analytics this application can offer relates to body image projections driven by sustained adherence to set benchmarks and lifestyle routine. These body image projections could be further enhanced by AI and integrated lifestyle monitoring devices for a more realistic historical base to project upon. In addition to the built-in positive reinforcement this application can offer, having the advantage to visualize “your future self” can add to the user's motivation and success rate.

Predictive analytics can further distinguish this application from its competitors. It can empower the user with timely information to assist with meal planning and serve as a motivational resource to accomplish set goals.

### CONCLUSION

Many other variations than those described herein can be apparent from this document. For example, depending on the embodiment, certain acts, events, or functions of any of the methods and algorithms described herein can be performed in a different sequence, can be added, merged, or left out altogether (such that not some or all described acts or events are necessary for the practice of the methods and algorithms). Moreover, in certain embodiments, acts or events can be performed concurrently, such as through multi-threaded processing, interrupt processing, or multiple processors or processor cores or on other parallel architectures, rather than sequentially. In addition, different tasks or processes can be performed by different machines and computing systems that can function together.

The various illustrative logical blocks, modules, methods, and algorithm processes and sequences described in connection with the embodiments disclosed herein can be implemented as electronic hardware, computer software, or combinations of both. To clearly illustrate this interchangeability of hardware and software, various illustrative components, blocks, modules, and process actions have been described above generally in terms of their functionality. Whether such functionality can be implemented as hardware or software depends upon the particular application and design constraints imposed on the overall system. The described functionality can be implemented in varying ways for each particular application, but such implementation decisions should not be interpreted as causing a departure from the scope of this document.

The various illustrative logical blocks and modules described in connection with the embodiments disclosed herein can be implemented or performed by a machine, such as a general purpose processor, a processing device, a computing device having one or more processing devices, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor and processing device can be a microprocessor, but in the alternative, the processor can be a controller, microcontroller, or state machine, combinations of the same, or the like. A processor can also be implemented as a combination of computing devices, such as a combination of a DSP and a microprocessor, a plurality of microprocessors, one or more microprocessors in conjunction with a DSP core, or any other such configuration.

Embodiments of the systems and methods described herein can be operational within numerous types of general purpose or special purpose computing system environments or configurations. In general, a computing environment can include any type of computer system, including, but not limited to, a computer system based on one or more microprocessors, a mainframe computer, a digital signal processor, a portable computing device, a personal organizer, a device controller, a computational engine within an appliance, a mobile phone, a desktop computer, a mobile computer, a tablet computer, a smartphone, and appliances with an embedded computer, to name a few.

Such computing devices can be typically be found in devices having at least some minimum computational capability, including, but not limited to, personal computers, server computers, hand-held computing devices, laptop or mobile computers, communications devices such as cell phones and PDA's, multiprocessor systems, microprocessor-based systems, set top boxes, programmable consumer electronics, network PCs, minicomputers, mainframe computers, audio or video media players, and so forth. In some embodiments the computing devices can include one or more processors. Each processor may be a specialized microprocessor, such as a digital signal processor (DSP), a very long instruction word (VLIW), or other microcontroller, or can be conventional central processing units (CPUs) having one or more processing cores, including specialized graphics processing unit (GPU)-based cores in a multi-core CPU.

The process actions of a method, process, or algorithm described in connection with the embodiments disclosed herein can be embodied directly in hardware, in a software module executed by a processor, or in any combination of the two. The software module can be contained in computer-readable media that can be accessed by a computing device. The computer-readable media includes both volatile and nonvolatile media that can be either removable, non-removable, or some combination thereof. The computer-readable media can be used to store information such as computer-readable or computer-executable instructions, data structures, program modules, or other data. By way of example, and not limitation, computer readable media may comprise computer storage media and communication media.

Computer storage media includes, but may not be limited to, computer or machine readable media or storage devices such as Blu-Ray™ discs (BD), digital versatile discs (DVDs), compact discs (CDs), floppy disks, tape drives, hard drives, optical drives, solid state memory devices, RAM memory, ROM memory, EPROM memory, EEPROM memory, flash memory or other memory technology, magnetic cassettes, magnetic tapes, magnetic disk storage, or other magnetic storage devices, or any other device which can be used to store the desired information and which can be accessed by one or more computing devices.

A software module can reside in the RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The processor and the storage medium can reside in an application specific integrated circuit (ASIC). The ASIC can reside in a user terminal. Alternatively, the processor and the storage medium can reside as discrete components in a user terminal.

The phrase “non-transitory,” in addition to having its ordinary meaning, as used in this document means “enduring or long-lived”. The phrase “non-transitory computer-readable media,” in addition to having its ordinary meaning, includes any and some or all computer-readable media, with the sole exception of a transitory, propagating signal. This includes, by way of example and not limitation, non-transitory computer-readable media such as register memory, processor cache and random-access memory (RAM).

Retention of information such as computer-readable or computer-executable instructions, data structures, program modules, and so forth, can also be accomplished by using a variety of the communication media to encode one or more modulated data signals, electromagnetic waves (such as carrier waves), or other transport mechanisms or communications protocols, and includes any wired or wireless information delivery mechanism. In general, these communication media refer to a signal that has one or more of its characteristics set or changed in such a manner as to encode information or instructions in the signal. For example, communication media includes wired media such as a wired network or direct-wired connection carrying one or more modulated data signals, and wireless media such as acoustic, radio frequency (RF), infrared, laser, and other wireless media for transmitting, receiving, or both, one or more modulated data signals or electromagnetic waves. Combinations of the any of the above should also be included within the scope of communication media.

Further, one or any combination of software, programs, computer program products that embody some or all of the various embodiments of the systems and methods described herein, or portions thereof, may be stored, received, transmitted, or read from any desired combination of computer or machine readable media or storage devices and communication media in the form of computer executable instructions or other data structures.

Embodiments of the systems and methods described herein may be further described in the general context of computer-executable instructions, such as program modules, being executed by a computing device. Generally, program modules include routines, programs, objects, components, data structures, and so forth, which perform particular tasks or implement particular abstract data types. The embodiments described herein may also be practiced in distributed computing environments where tasks are performed by one or more remote processing devices, or within a cloud of one or more devices, that are linked through one or more communications networks. In a distributed computing environment, program modules may be located in both local and remote computer storage media including media storage devices. Still further, the aforementioned instructions may be implemented, in part or in whole, as hardware logic circuits, which may or may not include a processor.

Conditional language, such as, among others, “can,” “might,” or “may,” unless specifically stated otherwise, or otherwise understood within the context as used, can be generally intended to convey that certain embodiments include, while other embodiments do not include, certain features, elements and/or steps. Thus, such conditional language can be not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Unless the context clearly requires otherwise, throughout the description and the claims, the words “comprise,” “comprising,” “include,” “including,” “having,” and the like are to be construed in an inclusive sense, as opposed to an exclusive or exhaustive sense; that can be to say, in the sense of “including, but not limited to.” As used herein, the terms “connected,” “coupled,” or any variant thereof means any connection or coupling, either direct or indirect, between two or more elements; the coupling or connection between the elements can be physical, logical, or a combination thereof. Additionally, the words “herein,” “above,” “below,” and words of similar import, when used in this application, refer to this application as a whole and not to any particular portions of this application. Where the context permits, words in the above Detailed Description using the singular or plural number may also include the plural or singular number respectively. The word “or” in reference to a list of two or more items, covers some or all of the following interpretations of the word: any one of the items in the list, some or all of the items in the list, and any combination of the items in the list. Likewise, the term “and/or” in reference to a list of two or more items, covers some or all of the following interpretations of the word: any one of the items in the list, some or all of the items in the list, and any combination of the items in the list.

Any claims intended to be treated under 35 U.S.C. § 112(f) can begin with the words “means for”, but use of the term “for” in any other context can be not intended to invoke treatment under 35 U.S.C. § 112(f). Accordingly, the applicant reserves the right to pursue additional claims after filing this application, in either this application or in a continuing application.

What is claimed is:

1. A streamlined nutrition tracking user interface system, the system comprising:
a memory device programmed with computer-executable instructions corresponding to a streamlined nutrition tracking application;
a hardware processor configured to execute the computer-executable instructions so as to execute the streamlined nutrition tracking application on a user device, the application configured to:
receive an input of a user's demographic parameters;
calculate a basic metabolic rate based on the input of the user's demographic parameters;
calculate an adjusted metabolic rate based on the input of the user's lifestyle parameters and norms;
output a nutritional user interface that comprises streamlined nutritional options derived from a database and one or more fields that enable the user to provide user food or beverage consumption input based on the streamlined nutritional options;
receive, from the nutritional user interface, user food or beverage consumption input based on the streamlined nutritional options;
request an adjustment of the user food or beverage consumption input based on aggregated machine learning of user food or beverage consumption inputs of a plurality of users and based on aggregated user specific performance against user set benchmarks; and
output nutritional performance data to the user based on the adjusted metabolic rate and the adjustment of the user food or beverage consumption input, the nutritional performance data comprising a graph or chart that indicates nutritional consumption of the user over time.

2. The system of claim 1, wherein the user's demographic parameters comprise at least one of: gender, weight, height, or age.

3. The system of claim 1, wherein the user's lifestyle parameters and norms comprise an estimated average level of exertion or frequency of physical activity.

4. The system of claim 1, wherein the user's lifestyle parameters and norms comprise data associated with at least one monitoring device comprising at least one of: a smart weight scale, physical activity monitor, or glucose monitor.

5. The system of claim 1, wherein the application is configured to receive a user's current fitness goal.

6. The system of claim 5, wherein the user's current fitness goal comprises at least one of: body building, weight maintenance, or fat loss.

7. The system of claim 5, wherein the application is configured to determine one or more nutrition benchmarks based on the user's current fitness goal.

8. The system of claim 7, wherein the user set benchmarks comprise one or more updated nutrition benchmarks based on user-generated modifications to the one or more nutrition benchmarks.

9. The system of claim 8, wherein the user-generated modifications are limited by the basic metabolic rate or adjusted basic metabolic rate.

10. The system of claim 1, wherein the graph or chart that indicates nutritional consumption comprises a moving average nutrition against the user set benchmarks.

11. The system of claim 1, wherein the application is configured to analyze the beverage or food consumption input to determine a projection of the user's body composition.

12. The system of claim 1, wherein the adjustment to the food or beverage consumption input comprises: a change in the type of food or beverage or a change in a portion size of the food or beverage.

13. The system of claim 1, wherein the application is configured to:
receive a user indication of an unknown food or beverage;
request information associated with the unknown food or beverage; and
determine an identity of the unknown food or beverage based on the requested information.

14. The system of claim 1, wherein the application is configured to determine a target caloric burn per user workout based on the adjusted basic metabolic rate.

15. A method for streamlined nutrition tracking, the method comprising:
receiving an input of a user's demographic parameters;
calculating a basic metabolic rate based on the input of the user's demographic parameters;
calculating an adjusted metabolic rate based on the input of the user's lifestyle parameters and norms;
outputting a nutritional user interface that comprises streamlined nutritional options derived from a database and one or more fields that enable the user to provide user food or beverage consumption input based on the streamlined nutritional options;
receiving, from the nutritional user interface, user food or beverage consumption input based on the streamlined nutritional options;
requesting an adjustment of the user food or beverage consumption input based on aggregated machine learning of user food or beverage consumption inputs of a plurality of users and based on aggregated user specific performance against user set benchmarks; and
outputting nutritional performance data to the user based on the adjusted metabolic rate and the adjustment of the user food or beverage consumption input, the nutritional performance data comprising a graph or chart that indicates nutritional consumption of the user over time.

16. The method of claim 15, wherein the user's demographic parameters comprise at least one of: gender, weight, height or age.

17. The method of claim 15, wherein the user's lifestyle parameters and norms comprise an estimated average level of exertion or frequency of physical activity.

18. The method of claim 15, wherein the user's lifestyle parameters and norms comprise data associated with at least one monitoring device comprising at least one of: smart weight scale, physical activity monitor, or glucose monitor.

19. The method of claim 15, comprising receiving a user's current fitness goal.

20. The method of claim 19, wherein the fitness goal comprises bodybuilding, maintenance, or fat loss.

21. The method of claim 19, comprising determining one or more nutrition benchmarks based on the user's current fitness goal.

22. The method of claim 21, wherein the user set benchmarks comprise one or more updated nutrition benchmarks based on user-generated modifications to the one or more nutrition benchmarks.

23. The method of claim 22, wherein the user-generated modifications are limited by the basic metabolic rate or adjusted basic metabolic rate.

24. The method of claim 23, wherein the graph or chart that indicates nutritional consumption comprises a moving average nutrition against the user set benchmarks.
